(19) United States
(12) Patent Application Publication
(10) Pub. No.: US 2019/0090838 A1
(43) Pub. Date: Mar. 28, 2019

(54) X-RAY FLUOROSCOPY AND IMAGING APPARATUS

(71) Applicant: SHIMADZU CORPORATION, Kyoto-Shi (JP)

(72) Inventors: YUKI TANAKA, Kyoto-Shi (JP); Atsuo Saito, Kyoto-Shi (JP)

(73) Assignee: SHIMADZU CORPORATION, Kyoto-Shi (JP)

(21) Appl. No.: 15/714,398

(22) Filed: Sep. 25, 2017

(51) Int. Cl. A61B 6/00; A61B 6/04

(52) U.S. Cl. CPC A61B 6/542; A61B 6/487; A61B 6/5205; A61B 6/0407; A61B 6/42; A61B 6/463; A61B 6/4441; A61B 6/40

(57) ABSTRACT

An X-ray fluoroscopy and imaging and apparatus prevents error in a foot switch having a sensor. The detection sensor detects whether the operator is contacting each switch installed on the foot-switch. The information detected by the sensor is displayed as a textual format and/or an image that are visually recognizable, so that the operator can confirm the accuracy of a contacted-switch indication. Accordingly, the operator can comprehend instantly and accurately information relative to the switch currently selected.

### CROSS REFERENCE TO RELATED APPLICATIONS

This application relates to, but does not claim priority from, JP 2015-058070 filed Mar. 20, 2015, the entire contents of which are incorporated herein by reference.

### FIGURE SELECTED FOR PUBLICATION

FIG. 3

### BACKGROUND OF THE INVENTION

### Field of the Invention

The present invention relates to an X-ray fluoroscopy and imaging apparatus to obtain an X-ray image of a subject and particularly relates to the X-ray fluoroscopy and imaging apparatus equipped with a foot-switch by which an operator enables to switch an on-off operation.

### Description of the Related Art

An X-ray fluoroscopy and imaging apparatus is used to implement an X-ray fluoroscopy or an X-ray imaging of a subject in a medical site. The X-ray fluoroscopy is for intermittently obtaining X-ray images by irradiating a relatively low-dose X-ray to the subject and being used for investigating an affected region. On the other hand, the X-ray imaging is for obtaining a clearer X-ray image by irradiating a relatively high-dose X-ray to the subject. Therefore, the X-ray fluoroscopy and the X-ray imaging are alternatively and frequently implemented by switching each operation while implementing an operative procedure.

It is known that a foot-switch is used to implement an input when X-ray fluoroscopy and imaging apparatus is being used. The operator operates medical instrument such as a catheter, a knife and so forth with both hands in many cases while implementing the operative procedure in the medical site. Then, the operator conducts the on-off operation of the X-ray fluoroscopy and the X-ray imaging by stepping on a foot-switch installed near by a foot of the operator. When operating the medical instrument with both hands, the operator enables to switch on-off between the X-ray fluoroscopy and the X-ray imaging at an adequate timing by using the foot-switch even when an input operation by a hand is difficult.

The foot-switch is also applied to the case when implementing an on-off operation to move the table on which the subject is loaded other than the case when implementing the on-off operation relative to the X-ray fluoroscopy and the X-ray imaging, and so forth. Accordingly, it is common that the foot-switch is equipped with a plurality of switches in a parallel arrangement. In such case, the operator steps on any selected from a plurality of switches and then implement the operation in accordance with the switch that is stepped on.

When operating by stepping on the foot-switch, the operator keeps looking at the hand by which the subject is loaded on the table and a medical instrument as a catheter is being operated or keeps watching the monitor displaying the X-ray image. Therefore, a trial aspect in which a plurality of switches can be discriminated without looking at the foot-switch under the foot has been conducted. Specifically, an aspect in which a divider in between the plurality of foot-switches is installed and an aspect in which each of switch shapes and forms is different from one another have been proposed.

In addition, recently an aspect in which an obstacle made of an elastic material is formed in the upper side of any one of a plurality of switches has been proposed (Patent Document 1). According to the conventional aspect disclosed in the Patent Document 1, as one example thereof, it is expected that the switch for X-ray fluoroscopy and the switch for X-ray imaging are discriminated based on with-or-without the obstacle that is installed upper side of the switch for the X-ray imaging. In addition, the force stepping on the foot-switch to operate the switch becomes stronger due to setting of the obstacle, so that an effect, on which it is avoidable that an X-ray imaging with an erroneously strong X-ray dose is implemented, has been expected.

### RELATED PRIOR ART DOCUMENTS

### Patent Document

Patent Document 1
JP2008-36278 A1

### ASPECTS AND SUMMARY OF THE INVENTION

According to one aspect of the present invention, there is provided an X-ray fluoroscopy and imaging and apparatus prevents error in a foot switch having a sensor. The detection sensor detects whether the operator is contacting each switch installed on the foot-switch. The information detected by the sensor is displayed as a textual format and/or an image that are visually recognizable, so that the operator can confirm the accuracy of a contacted-switch indication. Accordingly, the operator can comprehend instantly and accurately information relative to the switch currently selected.

### Objects to be Solved

Nevertheless, in the case of a conventional example having such structure, following problems are remained to be solved. Specifically, according to the conventional X-ray fluoroscopy and imaging apparatus, it is difficult to exactly discriminate a plurality of switches without casting an eye over the foot-switch. Therefore, it is concerned about problems in which a different operation from the exception is implemented by stepping on the wrong switch and consequently, the subject is exposed to the unwanted X-ray radiation and the required operative procedure takes a longer time, and so forth.

Accordingly, when the conventional foot-switch is used, the operator must often cast an eye over the foot-switch out of the monitor or the subject in order to absolutely avoid stepping on the wrong switch. In such case, it can be concerned about problems in which efficiency of the X-ray imaging and the X-ray fluoroscopy is impaired due to that the operator is hard to concentrate the line of sight on the monitor or a workload of the operator increases due to that the operator has to move frequently the line of sight.

The present invention is completed under considering such concerns, and the purpose of the present invention is to provide an X-ray fluoroscopy and imaging apparatus, in which the operator enables to absolutely avoid erroneously stepping on the switch without casting an eye over the foot.

### Means for Solving the problem

The present invention constitutes the following structure to solve such problems.

Specifically, an X-ray fluoroscopy and imaging apparatus of the present invention comprises: an X-ray tube that irradiates an X-ray to a subject; an X-ray detection element that detects an X-ray transmitting a subject and outputs a detection signal; a foot-switch that controls on-off of the X-ray irradiation from the X-ray tube by that an operator operates the foot-switch by a foot; a switch-operation detection means that detects that the operator is contacting the control switch or that the control switch is displaced somewhat at a degree of level in which the control switch is not turned on; and a switch-operation display means that displays a detection result detected by the switch-operation detection means.

### Action and Effect

According to the X-ray fluoroscopy and imaging apparatus of the present invention, the switch-operation detection means detects that the operator is contacting the control switch or that the control switch is displaced somewhat at the degree of level in which the control switch is not turned on. Subsequently, the switch-operation display means displays the result detected the switch-operation detection means. Consequently, the operator can recognize visually and comprehend that the operator is contacting the control switch or that the control switch is displaced somewhat at the degree of level in which the control switch is not turned on.

Eyesight is a keen sense, so that the operator can comprehend instantly and accurately the detection result detected by the switch-operation detection means. Specifically, the operator can comprehend instantly and accurately the information relative to that the operator is contacting the control switch or the information relative to that the control switch is displaced somewhat at the degree of level in which the control switch is not turned on. Therefore, an implementation of an operation that is different from the expected operation due to that the control switch erroneously is stepped on is absolutely avoided, so that unwanted radiation exposure to the subject can be prevented. In addition, the time elongation required for the operative procedure can be adequately avoided.

According to the present invention set forth above, it is preferable that the X-ray fluoroscopy and imaging apparatus comprises: an image generation element that generates an X-ray image based on the X-ray detection signal output from the X-ray detection element; and an image display element that displays the X-ray image generated by the image generation element; wherein the switch-operation display means is installed to the image display means.

### Action and Effect

According to the X-ray fluoroscopy and imaging apparatus of the present invention, the switch-operation display means is installed to the image display means. The image display means displays an X-ray image, so that the operator has to frequently and repeatedly cast an eye over the image display means to double-check the X-ray image in process of implementation of the operative procedure. When such constitution is operative, the operator can make sure the result, which the switch-operation detection detects, while casting the eye over the image display means. Therefore, the operator does not have to move the line of sight from the image display means in order to absolutely avoid erroneously stepping on the control switch, so that workload of the operator can be further reduced.

According to the present invention set forth above, it is preferable that the X-ray fluoroscopy and imaging apparatus comprises: an apparatus information detection means that detects the locational information of the X-ray tube and the directional information of X-ray irradiation as an apparatus information; and an apparatus information display means that displays the apparatus information, wherein the switch-operation display means is installed to the apparatus information display means.

### Action and Effect

According to the X-ray fluoroscopy and imaging apparatus of the present invention, the switch-operation display means is installed to the apparatus information display means that displays the apparatus information. In process of implementation of the operative procedure, the operator has to frequently and repeatedly cast the eye over the image display means to double-check the locational information of the X-ray tube and the directional information of X-ray irradiation. When the above constitution is operative, the operator can make sure the result detected by the switch-operation detection means under the condition in which the operator is casting the eye over the apparatus information display means. Therefore, the operator does not have to move the line of sight frequently to absolutely avoid erroneously stepping on the control switch, so that workload of the operator can be further reduced.

According to the present invention set forth above, it is preferable that an input element that inputs an X-ray irradiation condition relative to the X-ray tube; wherein the switch-operation display means is installed to the input element.

### Action and Effect

According to the X-ray fluoroscopy and imaging apparatus of the present invention, the switch-operation display means is installed to the input element. The operator has to frequently and repeatedly cast the eye over the input element to input the X-ray irradiation conditions of the X-ray tube in process of implementation of the operative procedure. When the above constitution is operative, the operator can make sure the result detected by the switch-operation detection means while casting the eye over the input element. Therefore, the operator does not have to move the line of sight frequently to absolutely avoid erroneously stepping on the control switch, so that workload of the operator can be further reduced.

According to the present invention set forth above, it is preferable that the control switch is a two-step switch by which the first-step to prepare an X-ray irradiation from the X-ray tube and the second-step to implement the X-ray irradiation are operatively implemented, and the switch-operation detection means discriminates and detects the state in which the operator is contacting the control switch, the state in which the operator is implementing the first-step operation relative to the control switch and the state in which the operator is implementing the second-step operation relative to the control switch.

### Action and Effect

According to the X-ray fluoroscopy and imaging apparatus of the present invention, the control switch is the two-step switch. Then, the switch-operation detection means discriminates and detects the state in which the operator is contacting the control switch, the state in which the operator is implementing the first-step operation relative to the control switch and the state in which the operator is implementing the second-step operation relative to the control switch.

The operator comprehends instantly and accurately which switch is being operated at which step by visually recognizing the information displayed on the switch-operation display means. Therefore, the operator can instantly make sure that the present state of X-ray irradiation from the X-ray tube is which one of the not-prepared state, the prepared state, and the implementing state; so that the operator can further absolutely avoid miscomprehension relative to the step in which the control switch is being operated. Consequently, the operator can adequately avoid the unwanted radiation exposure to the subject and the operation elongation required for the operative procedure.

According to the present invention set forth above, it is preferable that the switch-operation detection means is a distance meter that measures the distance between the operator's foot and the control switch.

### Action and Effect

According to the X-ray fluoroscopy and imaging apparatus of the present invention, the switch-operation detection means is the distance meter that measures detects the distance between the operator's foot and the control switch. The switch-operation detection means detects that the operator is contacting the control switch or that the control switch is displaced somewhat at the degree of level in which the control switch is not turned on, based on the distance between the control switch and the foot thereof. According to such aspect, the switch-operation detection means detects quickly the event, in which the operator is going to contact the control switch, at the point when the operator brings the foot thereof close to the control switch to control the control switch. Accordingly, the operator comprehends momentarily the result detected by the switch-operation detection means, so that the operator can proceed more instantly and accurately the operative procedure.

According to the present invention set forth above, it is preferable that the switch-operation detection mean is a pressure sensor that measures pressure force relative to the control switch.

### Action and Effect

According to the X-ray fluoroscopy and imaging apparatus of the present invention, the switch-operation detection means is the pressure sensor that detects the pressure force relative to the control switch. According to such aspect, the operator can absolutely detect that the operator is contacting the control switch or that the control switch is displaced somewhat at the degree of level in which the control switch is not turned on, based on the pressure being added to the control switch. Therefore, the operator enables to comprehend absolutely the detection result detected by the switch-operation detection means, so that the operative procedure can be implemented further instantly and accurately.

In addition, according to the aspect of the present invention, it is preferable that the foot-switch comprises a plurality of the control switches and the switch control operation display means displays a contact or a displacement relative to any of the control switches.

### Action and Effect

According to the X-ray fluoroscopy and imaging apparatus of the present invention, the foot-switch comprises a plurality of the control switches and the switch-operation display means displays that any of the control switches is contacted or displaced. The operator recognizes visually the information displayed by the switch-operation display means, so that the operator can momentarily and absolutely comprehend that any of the control switches is contacted or displaced. Therefore, the operator can absolutely avoid, any errant operation due to an operation of a different control switch from the wanted control switch, so that unwanted radiation exposure to the subject can be prevented. In addition, the time elongation required for the operative procedure can be adequately avoided.

### EFFECT OF THE INVENTION

According to the X-ray fluoroscopy and imaging apparatus of the present invention, the switch-operation detection means detects that the operator contacts the control switch or that the control switch is displaced somewhat at the degree of level in which the control switch is not turned on. Subsequently, the switch-operation display means displays the result detected by the switch-operation detection means. Consequently, the operator recognizes visually the switch-operation display means, so that the operator can comprehend through eyesight that the operator is contacting the control switch or that the control switch is displaced somewhat at the degree of level in which the control switch is not turned on.

Eyesight is a keen sense, so that the operator can comprehend instantly and accurately the result detected by the switch-operation detection means. Specifically, the operator can comprehend instantly and accurately the information relative to that the operator is contacting the control switch or that the control switch is displaced somewhat at the degree of level in which the control switch is not turned on. Therefore, an implementation of an operation that is different from the expected operation due to that the control switch is erroneously stepped on is absolutely avoided, so that unwanted radiation exposure to the subject can be prevented. In addition, the time elongation required for the operative procedure can be adequately avoided.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a schematic view illustrating the entire structure of an X-ray fluoroscopy and imaging apparatus according to the aspect to the Embodiment 1.

FIG. 2 is a functional block diagram illustrating the structure of the X-ray fluoroscopy and imaging apparatus according to the aspect to the Embodiment 1.

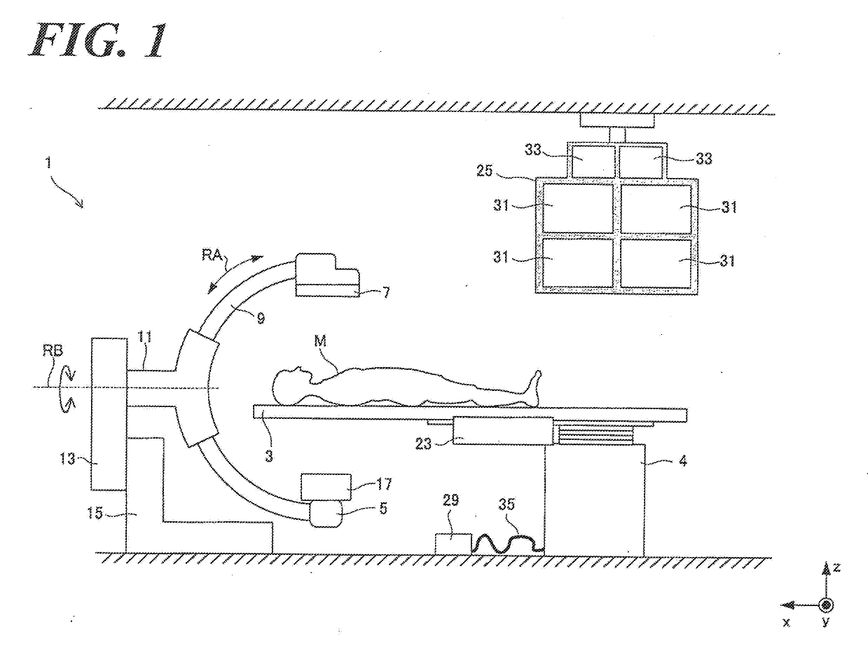

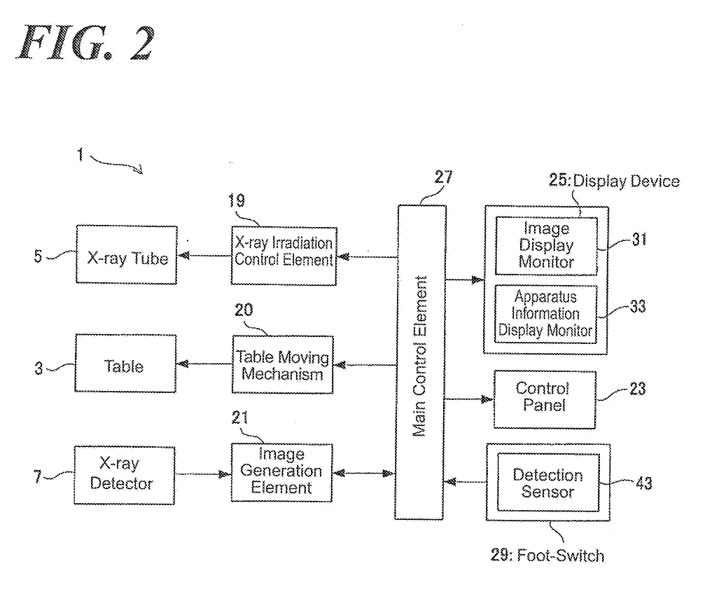

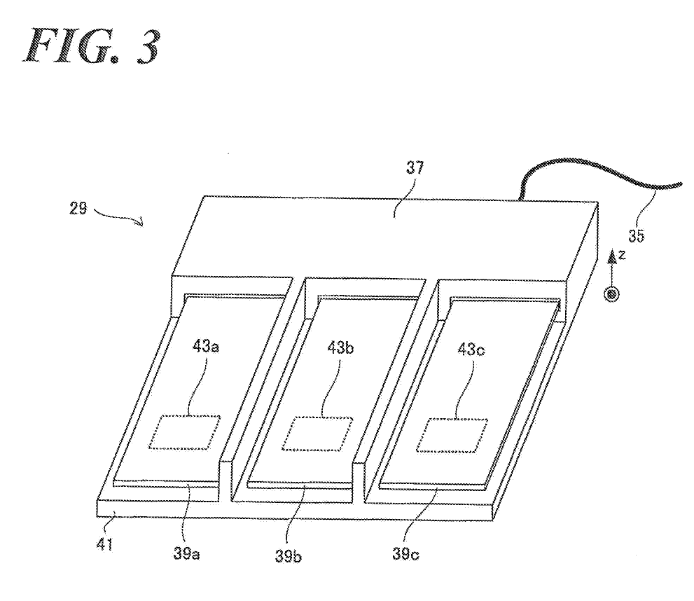

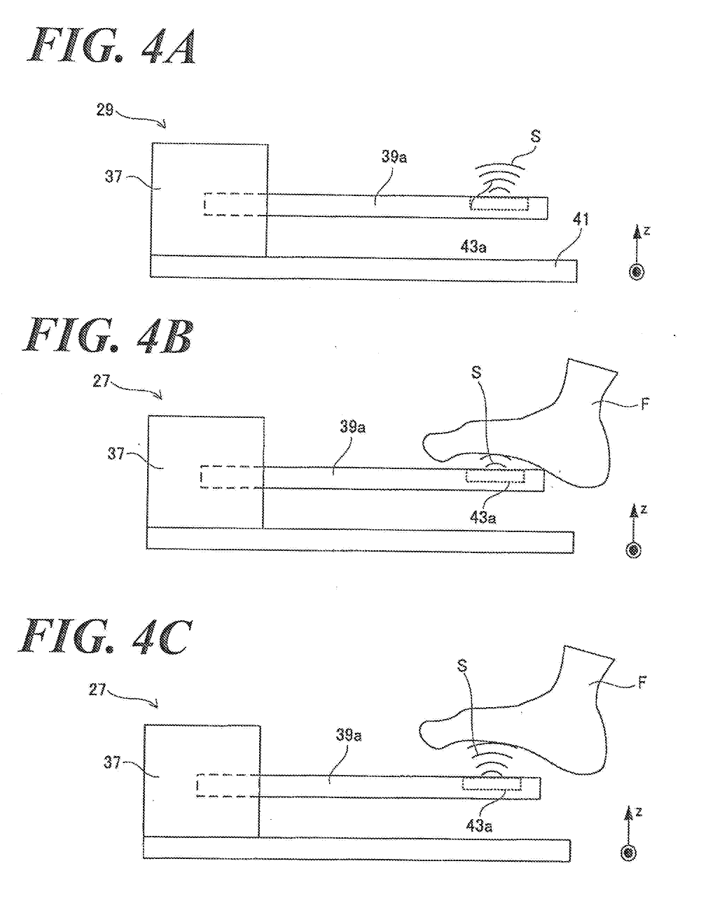

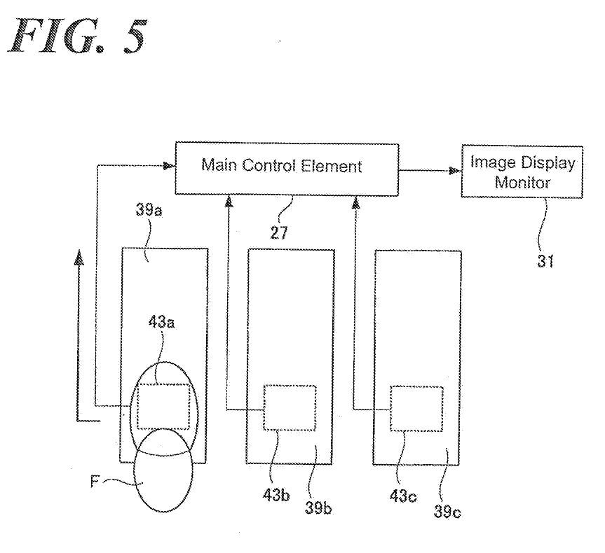

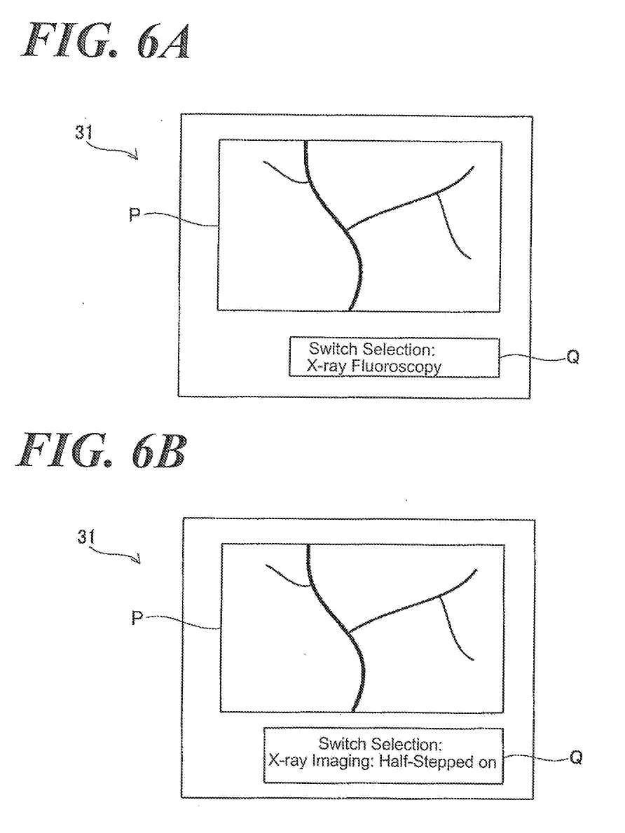

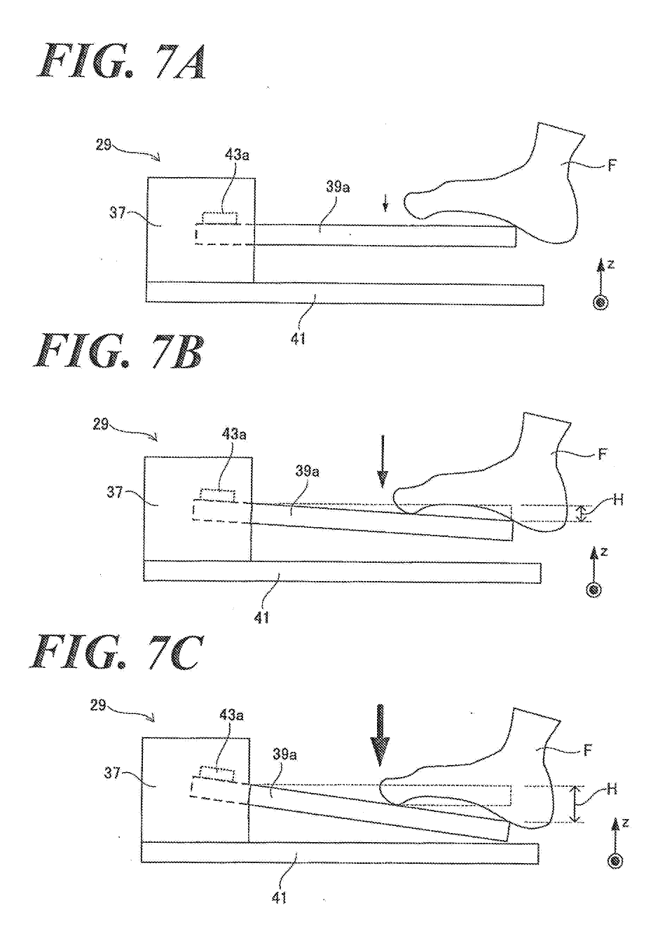

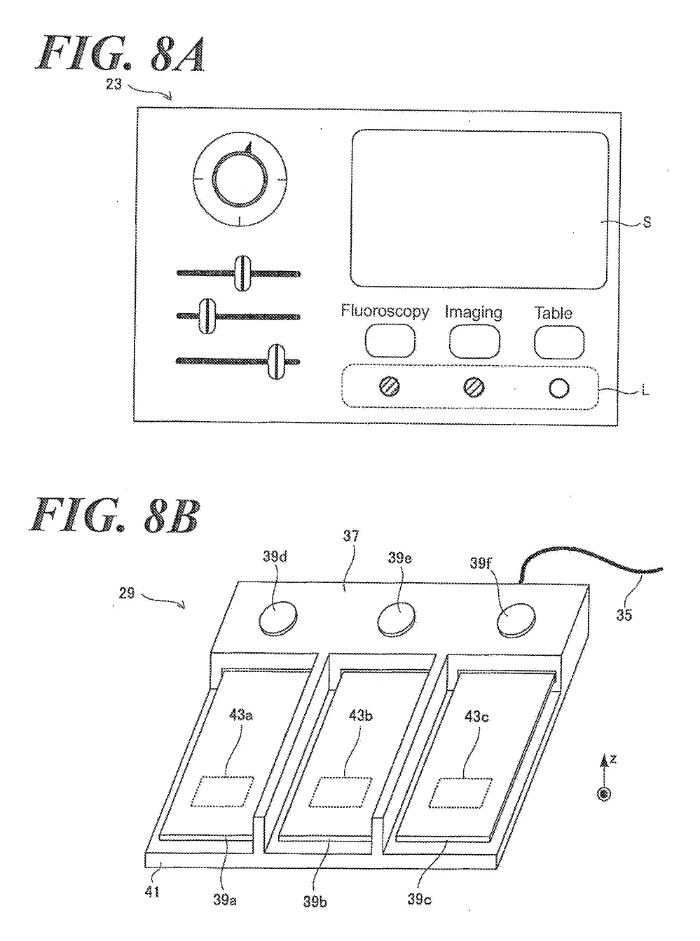

FIG. 3 is a perspective view illustrating the schematic structure according to the aspect to the Embodiment 1.

FIG. 4A-4C are illustrating the states in which the foot-switch according to the aspect of the Embodiment 1 is under operation.

FIG. 4A is a schematic view illustrating the operation state in which a foot is not close to the switching and the switch is not under operation.

FIG. 4B is the schematic view illustrating the operation state in which the foot is contacting the switch to be operated.

FIG. 4C is the schematic view illustrating the operation state in which the foot is close to the switch to be operated.

FIG. 5 is a schematic view illustrating the X-ray fluoroscopy and imaging apparatus according to the aspect of the Embodiment 1.

FIG. 6A, 6B are schematic diagrams illustrating the aspect in which the information relative to a selected switch is displayed.

FIG. 6A is the schematic diagram illustrating the information to be displayed on the image display monitor according to the aspect of the Embodiment 1.

FIG. 6B is the schematic diagram illustrating the information to be displayed on the image display monitor according to the aspect of the Embodiment 2.

FIG. 7A-7C are illustrating the states in which the foot-switch according to the aspect of the Embodiment 2 is under operation.

FIG. 7A is the schematic view illustrating the operation state in which the foot is contacting the switch to be operated.

FIG. 7B is the schematic view illustrating the state in which the switch is pressed half way.

FIG. 7C is the schematic view illustrating the state in which the switch is pressed completely.

FIG. 8A, 8B are illustrating the operation states according to the aspect of the alternative Embodiment is under operation.

FIG. 8A is a schematic diagram illustrating the aspect in which the information is displayed in the operation panel according to the aspect of the alternative Embodiment.

FIG. 8B is a perspective view illustrating the foot-switch according to the aspect of the alternative Embodiment.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

As used herein, any reference to a sensor or “computer-based device” or circuit for an input device for receiving data, an output device for outputting data in tangible form (e.g. printing or displaying on a computer screen), a permanent memory for storing data as well as computer code, and a microprocessor for executing computer code wherein said computer code resident in said permanent memory will physically cause said microprocessor to receive or read-in data via said input device, process said data within said microprocessor and output said processed data via said output device, such that one of skill in the computer, robotics, and imaging system arts, having studied the enclosed disclosure, will understand the aspects of the present invention.

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors and any combinations of these structures etc. as will be understood by those of skill in the art as also being identified as or capable of operating the systems and devices and subcomponents noted herein and structures that accomplish the functions without restrictive language or label requirements since those of skill in the art are well versed in related X-Ray diagnostic devices, computer and operational controls and technologies of radiographic devices and all their sub components, including various circuits and combinations of circuits without departing from the scope and spirit of the present invention.

### Embodiment 1

Referring to FIGs, the inventor sets forth the Embodiment 1 of the present invention. FIG. 1 is a schematic view illustrating the entire structure of an X-ray fluoroscopy and imaging apparatus according to the aspect to the Embodiment 1, and FIG. 2 is a functional block diagram illustrating the structure of the X-ray fluoroscopy and imaging apparatus according to the aspect to the Embodiment 1.

(Illustration of the Entire Structure)

An X-ray fluoroscopy and imaging apparatus according to the Embodiment 1 of the present invention 1 comprises a table 3 on which a subject M lies on horizontally, a supporting element 4 that supports the table 3, an X-ray tube 5 that irradiates an X-ray to the subject M, and an X-ray detector 7 that detects the X-ray irradiated from the X-ray tube 5 The table 3 is movable horizontally and vertically. The X-ray tube 5 and the X-ray detector 7 are in-place facing to each other sandwiching the table 3. The X-ray detector 7 comprises a detection surface that detects the X-ray, and X-ray detection elements are arranged two-dimensionally on the detection surface. The X-ray detector converts the detected X-ray to an electric signal and outputs the converted signal as an X-ray detection signal.

The X-ray tube 5 and the X-ray detector 7 are respectively installed to one end and the other end of the C-shape-arm (C-arm) 9. The C-arm 9 that is held by the arm holding member 11 is slidable along the circular arc pathway of the C-arm 9 indicated by the sign RA. The arm holding member 11 that is installed to the side place of the supporting post 13 is revolvable around the horizontal axis B parallel to the x-direction (longer direction of the table 3 and body axis direction of the subject M). The C-arm 9, which is held by the arm holding member 11, revolves around the axis of the x-direction along the arm holding member 11.

The supporting post 13 that is supported by the supporting base installed to the floor surface is movable horizontally in the y-direction (shorter direction of the table 3). The arm holding member 11 supported by the supporting post 13 and the C-arm 9 move in the y-direction following the horizontal move of the supporting post 13. A collimator 17 that is installed to the X-ray tube 5 limits X-rays irradiated from the X-ray tube 5 to a cone-shape like a pyramid.

The X-ray fluoroscopy imaging apparatus further comprises an X-ray irradiation control element 19, a table moving mechanism 20, an image generation element 21, an operation panel 23, a display device 25, a main control element 27 and a foot-switch 29. The X-ray irradiation control element 19 that is installed to the X-ray tube 5 outputs a high-voltage to the X-ray tube 5. The amount of X-ray irradiated by the X-ray tube 5 and the timing of X-ray irradiation are controlled based on the high-voltage output provided by the X-ray irradiation control element 19. The tabletop moving mechanism 20 that is connected to the tabletop 3 moves the tabletop 3 in the horizontal direction (x-direction and y-direction) and the vertical direction (x-direction). The image generation element 21 that is installed to the latter part of the X-ray detector 7 generates an X-ray image of the subject M based on the X-ray detection signal output from the X-ray detector 7.

The operation panel 23, to which an operator inputs a variety of directives, includes e.g., a keyboard, a mouse, or a touchpanel. The operator inputs the X-ray irradiation conditions, including such as movement of the table 3, a tube voltage of the X-ray tube 5 and the tube electric current, and the information to control the slide-move and the revolving move of the C-arm 9, by operating the input element 23. It is preferable that the operation panel 23 is in-place in the proximity of the operator, e.g., the side-place of the table 3, when the medical treatment is performed on the subject M. In addition, it is preferable that the location where the operation panel is installed is the place as high as the operator can manually operate when the medical treatment is performed. The operation panel 23 corresponds to the input element of the present invention.

The display device 25 comprises an image display monitor 31 and an apparatus information display monitor 33. The image display monitor 31 displays a variety of X-ray images generated by the image generation element 21. The apparatus information display monitor 33 displays physical information of each unit including the table 3, the X-ray tube 5 and the X-ray detector 7 constituting the X-ray fluoroscopy and imaging apparatus 1.

A specific example of the apparatus information includes information relative to three-dimensional coordinate location of each unit, information relative to the slide location of the C-arm 9 in the RA direction, and information relative to the revolving angle of the C-arm 9 in the RB direction and so forth. The number of the image display monitors 31 and the apparatus information display monitors 33 can be arbitrarily modified. In addition, it is preferable that the display device 25 is installed in the relatively high place so that the operator performing on the medical treatment can easily confirm a variety of information, and more preferably installed approximately at the height of the operator's eyes.

A plurality of sensors installed to the C-arm 9 and the arm holding member 11 detects respectively a slide-shift amount of the C-arm 9, a revolving-shift amount and horizontal shift amount of the arm holding element 11. The X-ray tube 5 and the X-ray detector 7 are installed to the C-arm 9, so that the respective spatial locations change following the shifts of the C-arm 9 and the arm holding member 11 while keeping facing arrangement state. In addition, the irradiation direction of the X-ray tube 5 changes in accordance with the shifts of the C-arm 9 and the arm holding member 11. Specifically, the locational information of the imaging system comprising the X-ray tube 5 and the X-ray detector 7 and the irradiation direction of the X-ray from the X-ray tube 5 are detected as the apparatus information based on each detection signal of the sensor. Each of sensors corresponds to the apparatus information detection means of the present invention.

The main control element 27 that comprises a central processing unit (CPU) and so forth controls comprehensively the X-ray irradiation control element 19, the table movement mechanism 20, the image generation element 21, the image display monitor 31 and the apparatus information display monitor 33 based on the directives input to the operation panel 23 and so forth. In addition, the main control element 27 that receives the information detected by the detection sensors set forth later sends the information to the image display monitor 31.

The foot-switch 29 that is placed on the floor under the table 3 connects the power supply via a cable 35 and the CPU (central processing unit) constituting the main control element 27. According to the aspect of the Embodiment 1, the main control element 27 is built into the support element 4 and in FIG. 1, the cable 35 is connected the support element 4. Referring to FIG. 3, the foot-switch 29 includes the body element 37, the foot-switches 39 that the operator operates by foot and the bottom plate 41. The switches 39 comprises three pedal-switches including a switch 39a for fluoroscopy, a switch 39b for imaging and a switch 39c for moving the table. According to the aspect of the Embodiment 1, the number of switches 39 are three, but the number of switches can be arbitrarily modified.

The switch 39a for fluoroscopy controls on-off relative to the X-ray fluoroscopy. Specifically, when the switch 39a for fluoroscopy is stepped on by foot, the main control element 27 sends the control signal to the X-ray irradiation control element 19. The X-ray control element 19 initiates an X-ray fluoroscopy in which relatively low-dose X-ray is irradiated intermittently from the X-ray tube 5. The switch 39b for imaging controls on-off relative to an X-ray imaging. Specifically, the X-ray irradiation control element 19 initiates an X-ray imaging in which relatively high-dose X-ray is irradiated from the X-ray tube 5 by stepping on the switch 39b for imaging.

The table movement switch 39c controls on-off of the movement of the table 3 in the vertical direction. Specifically, when the table movement switch 39c is stepped on by foot, a control signal is sent to a table movement mechanism 20 from the main control element 27, so that the table movement mechanism 20 changes the height of the table 3. The table 3 shifts in between the relatively low-position (loading position), at which the subject M gets on and off, and the relatively high-position (treatment position), at which the operator performs a medical treatment on the subject M in the decubitus position, in accordance with the operation of the table movement switch 39c.

Each of the switches 39 comprises a detection sensor 43. The detection sensor 43 detects that the switch 39 is contacted and sends the signal that identifies which sensor is contacted to the main control element 27. According to the Embodiment 1, the detection sensors 43 is an ultrasound distance meter that measures the distance to the obstacle therefrom by ultrasound (ultrasound sensor), but a known method relative to the detection sensor 43 can be arbitrarily applied as far as a contact is detected. For other examples, such detection sensor may include a pressure sensor and a photo sensor and so forth. Each of sensors 43 installed to the respective switches 39a-39c is distinguished as the respective sensors 43a-43c. The switch 39 corresponds to the control switch of the present invention. The detection sensor 43 corresponds to the switch-operation detection means of the present invention.

The inventor sets forth an example relative to the operation of the foot-switch 29 when the switch 39a for fluoroscopy is stepped on. Referring to FIG. 4A, when the switch 39a for fluoroscopy is not in use, ultrasonic wave S oscillates upward from the detection sensor 43a. Referring to FIG. 4B, when the operator touches the switch 39a for fluoroscopy by foot to operate the switch 39a for fluoroscopy, the upper side of the detection sensor 43a is blocked by the operator's foot F.

At this time. the switch 39a for fluoroscopy shifts somewhat in the z-direction without being turned on. The detection sensor 43a is blocked by the foot F, so that the ultrasonic wave reflects quickly from the operator's foot F. The detection sensor 43a measures the distance between the foot F and the detection sensor 43a based on the reflection time of the ultrasonic wave S and detects that the foot F is contacting the switch 39a for fluoroscopy.

Referring to FIG. 5, each of the detection sensors 43a-43b is connected to the main control element 27 and the main control element 27 is connected to the image display monitor 31. When the detection sensor 43a detects a contact of the foot F, a signal of the detail, which the operator's foot has contacted the switch 39a for fluoroscopy, is sent to the image display monitor 31 from the detection sensor 43a via the main control element 27.

Referring to FIG. 6, the image display monitor 31 displays the information Q, which identifies the switch, which the operator is contacting, based on the received signal. The information Q is displayed as the information, such as a textual information and an image information, that is visually recognizable. The operator can confirm that the switch 39 currently contacted is the switch 39a for fluoroscopy by visually recognizing the information Q. The information Q is visually recognizable, so that the operator can comprehend momentarily and absolutely that the operator is contacting the switch 39a for fluoroscopy. The image display monitor 31 corresponds to the switch-operation display means of the present invention.

The operator makes sure that the foot is currently contacting the switch 39a for fluoroscopy and steps on the switch 39a for fluoroscopy further toward the bottom plate 41. The switch 39a for fluoroscopy is stepped on, so that the X-ray fluoroscopy is turned on and subsequently, the relatively low-dose X-ray is intermittently irradiated from the X-ray tube 5. The image generation element 21 generates X-ray images (X-ray fluoroscopic images) intermittently by the X-ray fluoroscopy based on the X-ray detection signal output from the X-ray detector 7. The X-ray fluoroscopic image is displayed on the image display monitor 31.

When a clearer image than the X-ray fluoroscopic image is needed, the operator moves the position of the foot from the switch 39a for fluoroscopy to the switch 39b for imaging to perform the X-ray imaging by contacting the foot to the switch 39b for imaging. In such case, the detection sensor 43b detects that the operator's foot is contacting the switch 39b for imaging. Subsequently, the information Q indicating contact or displacement relative to which switch 39 is sent from the detection sensor 43b to the image display monitor 31 via the main control element 27. The image display monitor 31 displays the information Q. At this time, the detail of the information Q is the information demonstrating that the contacted or displaced switch out of the switch 39 is the switch 39b for imaging.

The operator visually recognizes the information Q displayed on the image display monitor 31 and makes sure that the foot is contacting the switch 39b for imaging. Then after, the operator steps on the switch 39b for imaging to turn on the state relative to the X-ray imaging, by which the relatively high-dose X-ray is irradiated from the X-ray tube 5. The image generation element 21 generates an X-ray image (X-ray radiograph) by the X-ray imaging based on the X-ray detection signal. The operator proceeds the operative procedure referring to the X-ray fluoroscopy image and the X-ray radiograph by switching the X-ray fluoroscopy and the X-ray imaging at an adequate and accurate timing while arbitrarily malting sure the switch being contacted by foot by visually recognizing the information Q.

In addition, when the operative procedure begins, the operator operates the switch 39c for moving the table to move vertically the table from the loading position to the treatment position following loading the subject M on the table 3 in the loading position. In such case, the detection sensor 43b detects that the foot F is contacting by contacting the operator's foot to the switch 39c for moving the table. Then, the detection sensor 43c sends the information Q, indicating that the switch 39 selected by the operator is the switch 39c for moving the table, to the main control element 27. Subsequently, the main control element 27 sends the information Q to the image display monitor 31 to be displayed.

The operator visually recognizes the information Q and makes sure instantly that the switch 39 which the operator per se is contacting is the switch 39c for moving the table, so that the operator can avoid to e.g., erroneously step on the switch 39b for imaging. Therefore, unwanted radiation exposure due to that the switch 39 is erroneously stepped on can be prevented. When the operative procedure ends, the operator operates the switch 39c for moving the table by foot to move vertically the table 3 from the treatment position to the loading position and unload the subject M from the table 3. Also, at this time, the detection sensor 43c detects that the operator is contacting the switch 39c for moving the table and subsequently, display the information Q, so that the operator can assuredly operates the switch 39c for moving the table.

In addition, in accordance with the Embodiment 1, it is preferable that the aspect applying such as an ultrasonic wave distance meter and an optical distance meter to a detection sensor 43 can display quickly the information Q based on the distance between the operator's foot and the detection sensor 43. Specifically, referring to FIG. 4C, when the foot F is close to the switch 39a for fluoroscopy, the distance between the detection sensor 43a and the foot F is shorter. The detection sensor 43a can detects that the operator would step on the switch 39a for fluoroscopy based on that the distance between the foot F and the detection sensor 43a is getting closer. Then, the detection sensor 43a displays the information Q, which is demonstrating that the switch 39 being contacted by the operator is the switch 39a for fluoroscopy, on the image display monitor 31 via the main control element 27 (refer to FIG. 6A).

According to the above aspect, even when the foot F is not contacting the switch 39, the detection sensor 43a detects the position of the foot F above the foot-switch 29 under the state in which the foot F is close to the switch 39a for fluoroscopy and sends the information relative to the detail of selecting the switch 39a for fluoroscopy. Accordingly, the operator can more quickly comprehend by eyesight that the switch, which the operator is going to select, is the switch 39a for fluoroscopy.

(Effects of the Aspect of the Embodiment 1)

According to a conventional foot-switch, the operator discriminates the switch to be stepped on by an aspect in which a divider in between the plurality of foot-switches is installed and an aspect in which each of switch shapes and forms is different from one another. Specifically, according to the conventional aspect, the operator comprehends the information relative to the contacted switch by only tactile (touching) sense. However, the tactile sense is relatively dull sense among five human senses, so that it is too hard to accurately discriminate the switch contacting the foot relative to the conventional foot-switch. Accordingly, the operator must look often at the foot-switch under the foot out of the line of sight toward the monitor or the subject in order to make sure which switch is currently selected. Consequently, not only the workload of the operator increases, but also time needed for proceeding the operative procedure is long.

On the other hand, the X-ray fluoroscopy and imaging apparatus 1 according to the Embodiment 1 comprises a foot-switch 29 having the detection sensor 43. The detection sensor 43 that is installed to each switch detects that the operator is contacting the switch 39 or that the control switch is displaced somewhat at the degree of level in which the control switch is not turned on. The switch 39 selected by the operator is identified which one of switches 39 installed to the foot-switch 29 by that the detection sensor 43 detects.

The result detected by the detection sensor 43 is displayed as a textual form and an image that are visually recognizable, so that the operator can make sure the information Q, identifying a switch contacted by foot, through eyesight. Eyesight is a keen sense, so that the operator can comprehend instantly and accurately the information Q identifying the switch currently selected.

In addition, the information detected by the detection sensor 43 is displayed on the image display monitor 31 having a different aspect from the foot-switch 29. When a medical treatment is implemented relative to the subject M, the line of sight of the operator is mainly focused on the image display monitor 31 to make sure the X-ray image P of the subject M. Referring to FIG. 6A, the image display monitor is in-place in the relatively high-location relative to the line of sight of the operator and the information Q is displayed on the image display monitor 31 together with the X-ray image P. Therefore, the operator can comprehend that the own foot is contacting the switch 39a for fluoroscopy, without moving the line of sight from the image display monitor 31 Accordingly, the operator can make sure further absolutely the information Q relative to the switch to be selected without losing concentration on the operative procedure.

In addition, a variety of sensors constituting the detection sensors 43 are all small, so that any sensor can be embedded in the foot-switch 29. Therefore, even when the detection sensors 43 are mounted, the problems due to glowing in size and increasing the cost of the foot-switch 29 can be avoid. In addition, the detection sensor 43 is embedded, so that the locational relationship between each of the switches 39 and each of the detection sensors 43 is the same as before. Therefore, even when the foot-switch 29 shifts, the detection sensors 43 absolutely detects the switch that is contacted by the operator and displays the accurate detail of the information Q. Accordingly, even when the foot-switch shifts due to an incident in which, for example, the operator erroneously kicks the foot-switch 29, the operator visually recognize the displayed information Q, so that the operator can make sure the accurate information of the switch 39 which the operator is contacting.

### Embodiment 2

Next, referring to FIGs, the inventors set so that the Embodiment 2 of the present invention. The entire structure of the X-ray fluoroscopy and imaging apparatus according to the aspect of the Embodiment 2 and the Embodiment 1 are in common. However, the detection sensor 43 according to the Embodiment 1 finds whether the operator's foot is contacting or not. On the other hand, the detection sensor 43 discriminates the respective states including the state in which the operator is not contacting the switch 39 (non-contact state), the state in which the operator's foot is contacting the control switch and the control switch is displaced somewhat (contact state), and the state in which the operator is stepping on the switch 39.

According to a general X-ray fluoroscopy and imaging apparatus, the switch that controls X-ray irradiation uses a two-step switch. Specifically, according to the aspect of e.g., the switch 39a for X-ray fluoroscopy, the switch 39a for X-ray fluoroscopy sends an X-ray irradiation preparation signal to the X-ray irradiation control element 19 via the main control element 27 under the state in which the switch 39a for X-ray fluoroscopy is stepped on until the first step (half-stepped-on state, referring to FIG. 7B). The X-ray control element 19 controls the X-ray tube subject to the X-ray irradiation preparation state, e.g., rotation of a target of the X-ray tube 5 according to the X-ray fluoroscopy preparation signal.

Specifically, according to the aspect of e.g., the switch 39a for X-ray fluoroscopy sends an X-ray irradiation preparation signal to the X-ray irradiation control element 19 via the main control element 27 at the state in which the switch 39a for X-ray fluoroscopy is stepped on until the first step (half-stepped-on state, referring to FIG. 7B). The X-ray control element 19 irradiates relatively low-dose X-ray from the X-ray tube 5 in accordance with an X-ray fluoroscopy implementation signal. While the non-contact state (FIG. 4A) and the contact state (FIG.4B, FIG. 7A) are discriminated according to the aspect of the Embodiment 1, non-contact state, contact-state, half-stepped-on state and full-stepped-on state are respectively discriminated according to the aspect of the Embodiment 2.

The inventor sets forth an example, in which the fluoroscopy switch 39a is stepped on, relative to the operation of the foot-switch 29 in accordance with the aspect of the Embodiment 2. According to the aspect of the Embodiment 2, a pressure sensor that detects a force added to each of the switches 39 is used.

Referring to FIG. 4A, in the non-contact state, no force is added to the foot-switch 39a for fluoroscopy. The detection sensor 43a sends the signal, indicating that the switches 39a for fluoroscopy is in the non-contact state, to the main control element 27 based on the event in which no force is added. On the other hand, in the contact state illustrated in FIG. 7A, a relatively weak force is added to the switch 39a for fluoroscopy. Then, when in the half-stepped-on state illustrated in FIG. 7B, the force larger than in the contact state is added to the switch 39a for fluoroscopy, and when in the full-stepped-on state illustrated in FIG. 7C, the further large force than in the half-stepped-on state is added to the switch 39a for fluoroscopy.

The detection sensor 43a finds that the switch 39a for fluoroscopy is in any state of the non-contact state, the contact state, the half-stepped-on state and the full-stepped-on state by detecting the force strength added to the switch 39a for fluoroscopy. The detection sensor 43a sends arbitrarily the signal, indicating that the switch 39a for fluoroscopy is in which state, to the main control element 27.

As well as the detection sensor 43a, the detection sensor 43b sends arbitrarily the signal indicating the state of the switch 39b for imaging to the main control element 27, and the detection sensor 43c sends arbitrarily the signal indicating the state of the switch 39c for moving the table to the main control element 27. The main control element 27 finds that the operator steps on which switch to which step (stage) based on the received information and sends the information Q, indicating the found detail, to image display monitor 31. Referring to FIG. 6B, the image display monitor 31 display the information Q under the condition in which the textual form and the image are visually recognizable.

The operator proceeds the operative procedure, referring to the information Q, by switching arbitrarily each operation of the X-ray fluoroscopy, the X-ray imaging and moving the table while visually recognizing the X-ray image P. For example, when the information Q illustrated in FIG. 6B is displayed, the operator visually recognizes the image display monitor 31, so that the operator can quickly comprehend that the own foot is stepping on the switch 39 for imaging to the first step and the preparation for X-ray imaging is completed. Therefore, according to the Embodiment 2, the operator can absolutely avoid an errant selection of the foot-switch 39 and an errant recognition of the step in which the switch 39 is stepped on without moving the line of sight to the foot-switch 29 under the foot.

In addition, the detection sensor 43 according to the Embodiment 2 includes e.g., a photo sensor and a micro-switch other than the pressure sensor. Specifically, referring to FIG. 7A, 7C, the switch 39a for fluoroscopy shifts in the z-direction, i.e., vertical direction following the operator steps on the switch 39 for fluoroscopy. Therefore, the detection sensor 43a finds each of the states including the non-contact state, the contact state, the half-stepped-on state and the full-stepped-on state by using the sensor detecting the moving distance H in the z-direction.

The present invention is not limited to the aspect of the Embodiments set forth above and another alternative Embodiment to be implemented set forth below.

(1) According to the aspect of each of above described Embodiments, the information Q that identifies the switch 39 operated by the operator is displayed on the image display monitor 31, but the target element to display the information Q is not limited to the image display monitor 31. Specifically, the information Q can be displayed on any structure that is installed to the place which is relatively high around above waist of the operator relative to the line of operator's sight differently from the switch 29 installed under the foot. In addition, a preferred target element to display the information Q is the apparatus information display monitor 33, the display device 25 or the operation panel 23 and so forth. In addition, the aspect to display the information Q is not limited to a textual format and an image.

Specifically, as one example, referring to FIG. 8A, a lamp L corresponding to each of switches 39 is installed to the operation panel 23 and each lump is arbitrarily turned on, so that the information Q can be displayed. Referring to FIG. 8A, the lamp L corresponding to the switch 39c for moving the table is turned on, so that the operator casts the eye over the operation panel 23 and therefore, the operator can comprehend that the switch 39c for moving the table currently is stepped on. In addition, the information Q in e.g., a textual format can be displayed on the panel S installed to the operation panel 23.

The operator frequently casts the eye over any of the apparatus information display monitor 33, the display device 25 and the operation panel 23 as well as the image display monitor 31 while proceeding the operative procedure. Specifically, the operator has to frequently and repeatedly cast the eye on the apparatus information display monitor 33 and the display device 25 to double-check the three-dimensional coordinate and the X-ray direction irradiated from the X-ray tube 5 to the X-ray detector 7. In addition, the operator frequently casts the eye on the operation panel 23 when inputs a variety of setting including the X-ray irradiation condition and when inputs a directive to run a variety of operations including movement of the C-arm 9 and so forth.

Accordingly, the information Q is being displayed on such components, so that the operator can comprehend that the operator is currently contacting the switch 39a for fluoroscopy without shifting the line of sight. In addition, the apparatus information display monitor 33, the display device 25 and the operation panel 23 are all installed to the relatively high-position to the operator's line of sight, which are different from the foot-switch 29. Accordingly, the operator does not have to shift the line of sight to the foot switch 29 under the own foot when makes sure the switch selected by the operator. Accordingly, the operator can make sure further absolutely the information Q relative to the switch 39 which the operator is currently stepping on without losing concentration on the operative procedure. Consequently, the operator can cut own workload and also the time needed for the operative procedure.

(2) According to each of above described Embodiments, the switch 39 installed to the switch 29 is not limited to a pedal-type switch. Specifically, the other kind of switch like a button-switch can be applied to each switch 39. In addition, referring to FIG. 8B, pedal-type switches 39a-39c and push-button-type switches 39d-39f can be installed side-by-side. In such aspect, the operator initiates an X-ray fluoroscopy by stepping on either the pedal-type switches 39a or the push-button-type switch 39d.

The detection sensors 43 are installed to each of the switches 39d-39F as well as the switches 39a-39c. Specifically, the operator contacts any of the switches 39d-39f by foot, so that the information identifying the switch contacted by the operator is sent to the image display monitor 31 via the main control element 27. Then, the image display monitor 31 displays the information, which identifies the switch contacted by the operator, among switches 39d-39f.

(3) According to the aspect of each Embodiment, the operations that are controlled by the switches 39 are the X-ray fluoroscopy, the X-ray imaging and the vertical moving of the table 3, but the operation that is controlled by the switch 39 is not limited thereto. The other example of the operation controlled by the switch 39 includes an on-off operation that controls a fixing mechanism of the position of the table 3. Specifically, when the switch 39 that controls locking of the table 3 is stepped on by foot, the fixing mechanism of the table 3 is unlocked, so that the table 3 becomes movable. In addition, the foot-switch 29 does not have to include the switch, such as the switch 39a for fluoroscopy, that controls the X-ray tube 5. Specifically, the switch to control the operation of the table 3, as the switch 39c for moving the table, can be installed.

(4) According to the aspect of each Embodiment, the inventor sets forth the example in which the X-ray fluoroscopy and imaging apparatus comprises a C-arm 9, but the aspect of the present invention can be applied to the system that does not have the C-arm. In such case, a sensor that detects a three-dimensional position coordinate of the X-ray tube 5 and an X-ray irradiation direction of the X-ray tube 5 as an apparatus information is installed to an adequate location. The detected apparatus information is sent to the main control element 27 and the apparatus information display monitor 33.

(5) According to the above Embodiment 2 set forth above, the respective switches 39 are a two-step switch, but each of the switches 39 can be a multi-step switch having at least three steps. In such case, each of the detection sensors 43 discriminates and detects the stepping-on state relative to the respective steps, and then send the detected information to the image display monitor 33.

### REFERENCE OF SIGNS

1 X-ray fluoroscopy and imaging apparatus
5 X-ray tube
2 Image generation element
23 Operation panel (input element)
25 Display devise
27 Main control element
29 Foot-switch
31 Image display monitor (switch operation display means)
33 Apparatus information display monitor
39 Switch (control switch)
43 Detection sensor (switch-operation detection means)

Although only a few embodiments have been disclosed in detail herein, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art.

Those of skill would further appreciate that the various illustrative logical blocks, modules, circuits, and algorithm steps described in connection with the embodiments disclosed herein may be implemented as electronic hardware, computer software running on a specific purpose machine that is programmed to carry out the operations described in this application, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments.

The various illustrative logical blocks, modules, and circuits described in connection with the embodiments disclosed herein, may be implemented or performed with a general or specific purpose processor, or with hardware that carries out these functions, e.g., a Digital Signal Processor (DSP), an Application Specific Integrated Circuit (ASIC), a Field Programmable Gate Array (FPGA) or other programmable logic device, sensor, discrete gate or transistor logic, discrete hardware components, or any combination thereof designed to perform the functions described herein. A general purpose processor may be a microprocessor, but in the alternative, the processor may be any conventional processor, controller, microcontroller, or state machine.

A processor or sensor with a processor may also be implemented as a combination of computing devices, e.g., a combination of a DSP and a microprocessor, a plurality of microprocessors, one or more microprocessors in conjunction with a DSP core, or any other such configuration to effectuate the present invention. These devices may also be used to select values for devices as described herein.

The steps of a method or algorithm described in connection with the embodiments disclosed herein may be embodied directly in hardware, in a software module executed by a processor, using cloud computing, or in combinations. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of tangible storage medium that stores tangible, non-transitory computer based instructions. An exemplary storage medium is coupled to the processor such that the processor can read information from, and write information to, the storage medium. In the alternative, the storage medium may be integral to the processor. The processor and the storage medium may reside in reconfigurable logic of any type.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and conununication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

Also, the inventors intend that only those claims which use the words “means for” are intended to be interpreted under 35 USC 112, sixth paragraph. Moreover, no limitations from the specification are intended to be read into any claims, unless those limitations are expressly included in the claims.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. An X-ray fluoroscopy and imaging apparatus, comprising:
an X-ray tube that irradiates an X-ray radiation to a subject;
an X-ray detector that detects an X-ray transmitting said subject and outputs an X-ray detection signal;
a foot-switch provided with a control switch that controls an on-off of said X-ray irradiation from said X-ray tube when an operator operates said control switch by foot; and
a switch-operation detector that detects that said operator is contacting said control switch or that said control switch is displaced at a level which said control switch is not turned on; and further comprising:
a switch-operation display that displays a result that is detected by said switch-operation detector.

2. The X-ray fluoroscopy and imaging apparatus, according to claim 1 further comprising:
an image generator that generates an X-ray image based on said X-ray detection signal output from said X-ray detector;
an image display that displays an X-ray image generated by said image generator; and
wherein said switch-operation display is installed to said image display.

3. The X-ray fluoroscopy and imaging apparatus, according to claim 1, further comprising:
an apparatus information detector that detects a locational information of said X-ray tube and a directional information of X-ray irradiation as an apparatus information;
an apparatus information display that displays said apparatus information; and
wherein said switch-operation display is installed to said apparatus information display.

4. The X-ray fluoroscopy and imaging apparatus, according to claim 1, further comprising:
an inputter that inputs an X-ray irradiation condition of said X-ray tube;
wherein said switch-operation display is installed to said inputter.

5. The X-ray fluoroscopy and imaging apparatus, according, to claim 1 wherein:
said control switch is a two-step switch by which a first-step to prepare an X-ray irradiation from said X-ray tube and a second-step to implement said X-ray irradiation from said X-ray tube, and
said switch-operation detector discriminates and detects a first state in which said operator is contacting said control switch, a second state in which the operator is implementing said first-step operation relative to said control switch and a third state in which said operator is implementing said second-step operation relative to said control switch.

6. The X-ray fluoroscopy and imaging apparatus, according to claim 1, wherein:
said switch-operation detector is a distance meter that measures a distance between a foot of said operator and said control switch.

7. The X-ray fluoroscopy and imaging apparatus, according to claim 1, wherein:
said switch-operation detector is a pressure sensor that measures a pressure force to said control switch.

8. The X-ray fluoroscopy and imaging apparatus, according to claim 1, wherein:
said foot-switch further comprises:
a plurality of said control switches; and
and said switch-operation display displays an indication when any of said control switches is contacted or displaced.
